(19) United States
(12) Patent Application Publication
(10) Pub. No.: US 2005/0074803 A1
(43) Pub. Date: Apr. 7, 2005

(54) Method for determining an active agent dose

(75) Inventors: Walter Schmitt, Neuss (DE); Stefan Willmann, Dusseldorf (DE); Edgar Diessel, Koln (DE); Ingmar Dorn, Koln (DE); Jens Burmeister, Koln (DE)

Correspondence Address: Norris, McLaughlin & Marcus P.A., 875 Third Avenue, 18th Floor, New York, NY, 10022, US

(73) Assignee: Bayer Technology Services GmbH, Leverkusen (DE)

(21) Appl. No.: 10/947,982

(22) Filed: Sep. 23, 2004

(30) Foreign Application Priority Data
Oct. 2, 2003 (DE) 103 45 837.9

(51) Int. Cl. C12Q 1/68; G06F 17/60; G06F 19/00; G01N 33/48; G01N 33/50

(52) U.S. Cl. 435/6; 702/20; 705/3

(57) ABSTRACT

Method for determining the dose of at least one active agent based on a genetic analysis. The method comprises analyzing specific genes for their nucleotide sequence or their expression levels of gene-specific proteins and/or RNA molecules. The gene-specific data is assigned one or more relevant physiological functions of the human or animal body, in particular those which have an influence on the metabolism, absorption, excretion or distribution of the active agent in the body. The gene-specific data and the assigned physiological functions are then integrated into a physiology-based pharmacokinetic model (PBPK model). The PBPK model integrates pharmacokinetic data relating to one or more active agents. The PBPK model may also receive and evaluate patient-specific data directly inputted and combined with data from a knowledge database comprising known values of pharmacokinetic parameters. The PBPK's integration of these data provide a calculation of the individual dose of the active agent.

The invention relates to a method for determining the individual dose of pharmaceuticals, for which it is known that their effect is influenced by pharmacokinetics and/or pharmacodynamics dependent on individual factors of the patients. Depending on the embodiment, the method may be employed either as a point of care solution directly in the clinic or medical practice, or as a special method in the field of laboratory medicine.

The therapeutic effect of medications is determined both by the intrinsic biochemical effect of the active agent directly on the biological target molecule and by the concentration at the site of action. The concentration at the site of action is in turn affected by various factors, such as the fraction absorbed in oral administration, the distribution in the body and the rate of metabolic breakdown and excretion. These processes depend greatly on the physiological and anatomical properties of the body of the patient being treated. Specifically, the following may be mentioned:
Volume and fat content of the individual organs or, as combined parameters, the body weight and the body fat content.
Blood flow rates in the individual organs.
Function of the gastrointestinal tract.
Function of the excretion organs such as the kidneys or biliary tract.
Expression and function of breakdown enzymes, particularly in the liver and intestines.
Expression and function of proteins for the active transport of molecules through cell membranes.

Since all these properties can vary from individual to individual owing to genetic predisposition, state of health or the influence of other medications, and therefore also on the concentration of the active agent in the body, individual differences are also encountered in the effect of medications. These may differ in degree depending on the properties of the active agent. It is known that, for most active agents, a therapeutic effect cannot be achieved in 50% of applications. The current procedure at the start of a therapy involves administration of the standard dose and subsequent observation of the patient. If a therapeutic result does not occur, attempts may be made to achieve it by gradually increasing the dose. This procedure is ineffective and may place the patient at risk. The latter is true especially for cases in which, owing to individual factors, the concentration in the body turns out to be substantially higher than in the normal case so that undesired side-effects take place.

The relationship between the individual properties of the body and the behaviour of a pharmaceutical active agent is at least qualitatively known in many cases. The specifics of many influencing factors, such as body weight or blood flow rate, can be diagnosed easily by the doctor in charge (weight) or estimated from medical knowledge, for example through changes in the perfusion when there is a disease. The influence of the body weight is compensated for in many cases by administering a weight-specific dose. In principle, however, current methods only allow qualitative adaptation to the individual situation.

The case regarding the influence of active biochemical processes is substantially more complicated. Here, the effectiveness of the processes may be modified if different amounts of the relevant proteins are present owing to genetic predisposition, disease or external influences, for example other active agents. Even with the same expression, the function of proteins may also be influenced for example by genetically induced alteration of the protein structure or by interaction with other substances (cf.: J. Licinio, M. Wong (Eds.), “Pharmacogenomics”. Wiley-VCH, Weinheim 2002; A. D. Rodrigues, “Drug-Drug Interactions”, Marcel Dekker, 2002).

The interaction with other substances, in particular other active agents administered at the same time or food ingredients, is primarily substance-dependent and, if the substances are characterized sufficiently, can be predicted at least qualitatively. For the most important proteins, there are lists of substances which influence their function either directly or by induction or inhibition of expression (cf.: A. Schinkel, J. W. Jonker “Mammalian drug efflux transporters of the ATP binding cassette (ABC) family: an overview), Advanced Drug Delivery Reviews 55, 3-29 (2003); 1 Cytochrome P450 Drug Interaction Table: http://medicine.iupui.edu/flockhart). In general, influences on the active agent in question are studied and noted in the form of a contraindication in the product information.

The effect due to the protein composition differing in respect of structure and amount in a particular patient's various organs at the time of therapy are more difficult to deal with. To that end, in principle, the expression of a particular protein in the organ in question needs to be determined, which is not generally possible since appropriate tissue samples are not available. Nowadays, however, it is known that differences in expression with respect to both the amount expressed and the protein structure are attributable, inter alia, to point mutations (replacement of individual nucleotides in the genomic DNA, so-called single nucleotide polymorphisms “SNPs”). SNPs may be found in the DNA sequences coding for the protein or in those regulating the RNA transcription and therefore the expression of the protein. Other types of mutations (for example insertions, deletions) which can modify the expression of proteins are also known. It is furthermore known that the methylation pattern superimposed on the genomic DNA can modify the transcription/expression. Such genomic markers, for example SNPs, can be detected at the DNA level in blood or other body fluids. In various cases, methods specially designed for this detection are already in development or available on the market, for example biochips or PCR-based detection methods. This opens up the opportunity for matched dosing based on a corresponding DNA test, directly in the medical practice or as a laboratory method. Information about the associated gene sequences, and modifications thereof, occurring in humans are available for many ADME-relevant proteins (cf.: SNP database on the Internet available at: http://www.ncbi.nlm.nih.gov/SNP/). For a large part, the effect of the individual modifications on the ADME-relevant function is also known (cf.: R. G. Tirona, R. B. Kim “Pharmacogenomics of Drug Transporters” in J. Licinio, M. Wong (Eds.), “Pharmacogenomics”. Wiley-VCH, Weinheim 2002).

One crucial problem in determining the optimum individual dose is the simultaneous complex dependency of the intracorporeal concentration on various influencing factors. While an individual dependency can still be experimentally determined and, for example, may be used in table form for a dosage decision, this is in general at most qualitatively possible when there are a plurality of dependencies influencing one another. This problem, however, can be resolved by using a computer-aided simulation to calculate the concentrations. One method suitable for this is the so-called physiology-based pharmacokinetic simulation (PBPK simulation), by which the absorption, distribution, metabolism and excretion (ADME) of xenobiotics in the mammalian body can be described in detail on the basis of physiological assumptions. For simple questions, which only take into account passive distribution processes in the body, this method has been known for a long time and extensively described (cf.: G. E. Blakey, I. A. Nestorov, P. A. Arundel, L. A. Aarons, M. Rowland “Quantitative Structure-Pharmacokinetics Relationships: 1. Development of a Whole Body Physiologically Based Model to Characterize Changes in Pharmacokinetics Across a homologous Series of Barbiturates in the Rat”, J. Pharmacokin. and Biopharm. 25, 277-312 (1997); R. Kawai, M. Lemaire, J.-L. Steiner, A. Bruelisauer, W. Niederberger, M. Rowland, “Physiologically Based Pharmacokinetic Study on a Cyclosporin Derivative, SDZ IMM 125”, J. Pharmacokin. and Biopharm. 22, 327-365 (1994)). A few known models also describe the metabolism and even the active transport in various organs as saturable non-dose-linear processes. A simulation model which takes the description of such processes into account throughout, in all the relevant organs of the body, is used in the PBPK simulation software PK-Sim (Bayer Technology Services GmbH).

The invention described here relates to a system comprising the combination of a detection system for determining the patient's ADME-relevant genetic predisposition, a PBPK/PD simulation and a database for substance properties (FIG. 1), which is suitable for dose-relatedly calculating the individual concentration in the body and proposing the optimum individual dose from the result.

The invention relates to a method for determining the dose of at least one active agent on the basis of a genetic analysis, having the following steps:
a) analysis (101) of specific gene sequences by means of a gene sequence-specific analysis instrument, in particular a sequence-specific sensor, or determination of the expression of proteins through either RNA transcription by means of quantitative RNA-specific detection methods or direct measurement of the protein expression by a protein analysis instrument,
b) allocation of the gene sequences to physiological functions of the human or animal body, in particular those physiological functions which have an influence on the metabolism, absorption, excretion or distribution of the active agent in the body,
c) delivery of the genetic data and allocation data to a physiology-based pharmacokinetic model (PBPK model) (108),
d) input of active agent-specific data into the PBPK model (108),
e) input of characteristic patient data, optionally from direct measurements on the body or into a computer system (104),
f) calculation of influencing physiological parameters necessary for the PBPK model from the patient data by using information contained in the knowledge database, and delivery of the parameters to the PBPK model (108),
g) calculation of the individual dose from the data according to steps c), d) and f) by using the PBPK model (108).

The patient's genetic predisposition in respect of the genes or proteins important for the ADME behaviour of active agents is determined by the gene test method (101).

A method is preferred in which the gene sequences are selected from those which relate to the proteins in the list:
metabolizing enzymes, in particular monooxygenases of the cytochrome P 450 family, phase II enzymes which attach polar groups to the molecules to be excreted, active transporters, in particular multidrug resistance proteins, for example the P-glycoprotein family or multidrug resistance-associated proteins (MRP) or the organic anion transporting polypeptide family (OATP) or the organic anion transporter family (OAT) or the organic cation transporter family (OCT) or the novel organic cation transporter family (OCTN) or the peptide transporter family (PepT), or plasma binding proteins, in particular serum albumin and glycoproteins.

The active agent-specific data are particularly preferably those selected from the list:
organ/blood distribution coefficients, membrane permeability, kinetic constants of the metabolism processes and/or of the active transport processes.

The characteristic patient data according to step e) are particularly preferably selected from the list:
body weight, body surface area, body fat content, age or sex, physiological functions departing from the normal state, for example owing to disease, for example renal or hepatic functional insufficiencies, co-medication.

The influencing physiological parameters according to step f) are particularly preferably selected from the list:
flow rate Qx of blood through the organ X, volume Vx of the organ X or permeability surface-area product (PxSAx) for the organ X.

The PBPK model is preferably a simulation program which simulates at least the following functions: intestinal absorption blood transport, distribution in organs by permeation or active transport, metabolism, excretion through urine or bile.

The invention also relates to a device for determining the dose of active agents, in particular by using the method according to the invention as described above, having at least one gene sequence-specific analysis instrument (101), a computer unit connected thereto with a program comprising a pharmacokinetic model (108), a knowledge database (105) and input modules (104) for patient data, characterized in that the PBPK model (108) is used as the pharmacokinetic model (108). The most important ADME-relevant proteins are:
Metabolizing enzymes: monooxygenases of the cytochrome P 450 family, phase II enzymes which attach polar groups to the molecules to be excreted.
Active transporters: multidrug resistance (P-glycoprotein family) (MDR), multidrug resistance-associated proteins (MRP), the organic anion transporting polypeptide family (OATP), the organic anion transporter family (OAT), the organic cation transporter family (OCT), the novel organic cation transporter family (OCTN) or the peptide transporter family (PepT).
Plasma binding proteins: in particular serum albumin and glycoproteins

Variations in the genetic coding, which occur with varying frequency and have a varyingly strong influence on the function of the proteins and therefore the ADME behaviour of an active agent, are known for many of these proteins.

Besides direct influence via ADME-relevant proteins, however, influence is also possible through genetically induced pathological states, which indirectly influence a process that is important for the ADME behaviour. Such genetic predispositions may also be included in the gene study, if the relationship with the active-agent behaviour is studied and can be described in the PBPK/PD model.

The gene test method (101) itself may, for example, be a method for directly determining the expression of the relevant proteins in the organ tissue, the transcription of relevant RNA molecules or alternatively a method for detecting SNPs of the DNA from samples of body fluids. A biochip- or PCR-based application is preferably involved in this.

The results of the gene test are evaluated by using a test-specific method (102) in order to obtain the necessary information about the influence on ADME-relevant processes. For instance, either the expression level of the proteins are determined directly or, in the case of DNA analysis, the effect on the function or expression of the corresponding protein is accordingly determined through known relationships. Genomic markers, for example SNPS, may also be used to classify patients into particular groups, for example fast or slow metabolizing patients. Attempts are also currently being made to find genomic markers which make it possible to classify patients into responders/non-responders or patients with and without expected side-effects in relation to particular medications or groups of medications. The data record (103) obtained in this way is sent as input data to the PBPK/PD model (108).

Further patient-specific data which are relevant to the dose calculation (104) are to be entered manually. These data involve information which can be found by measurement, exploration or anamnesis. Some examples are: body weight or body surface area, body fat content, age, sex. The parameter values of the PBPK/PD model which are obtained on the basis of these data are calculated in a subsequent step (106) with the aid of a knowledge database (105) through the fundamental relationships. This knowledge database may, for example, also contain information about the influence of particular diseases on ADME-relevant processes.

One possible embodiment of the module for manual input of the patient data could be an input device with a menu-driven user interface, which asks for other necessary information in a dynamically adapted way as a function of the entered information.

The active agent-specific data for the medication to be administered, which are needed for simulation of the ADME behaviour, are stored in another database (107). These data involve the parameter values, contained in the PBPK/PD model, which depend on the physico- and biochemical properties of the active agent. These have been determined beforehand, by direct experiment or through adapting the simulation model to pharmacokinetic and/or pharmacodynamic data. Examples of these data are organ/blood distribution coefficients, membrane permeabilities and the kinetic constants of the metabolism processes and of the active transport processes.

The central unit of the system is the PBPK/PD simulation model by which the actual calculation of the intracorporeal concentrations is carried out. The typical structure of a PBPK/PD model is shown in FIG. 2. The basic procedure is for the body to be subdivided into individual compartments and for the exchange of active-agent substance between these compartments to be described with the aid of conservation of mass equations. The individual organs are expediently selected as compartments.

Possibly, parts of the organs may also need to be defined as subcompartments, either if the substance transport between them may be limited or if information about concentration needs to be obtained separately.

These conservation of mass equations are ordinary differential equations. They typically have the form:

$${V}_{x} \cdot \frac{ⅆ {C}_{x}}{ⅆ t} = {Q}_{x} \cdot {C}_{ar} - {Q}_{x} \cdot \frac{{C}_{x}}{{K}_{x}} \qquad ( Equation 1 )$$

Vₓ=volume of the organ X
Cₓ=concentration of the substance in the organ X
Qₓ=flow rate of blood through the organ X
Cₐᵣ=concentration of the substance which reaches the organ via the arterial blood
Kₓ=distribution coefficient of the substance between blood and organ X in the equilibrium state

They describe the change of the concentration in the organ X due to the amount transported into the organ by the blood flow Qx, the distribution between blood and organ tissue, determined by the distribution coefficient Kx, and the amount transported away from the organ again by the blood flow.

For many pharmaceutical active agents, the distribution into the individual organs is limited since they permeate through the cell membranes more slowly than they are transported into the organ via the blood. In this case, the organs are to be divided into various subcompartments, which are separated from one another by membranes, and a model corresponding to FIG. 3 is obtained. The subcompartments to be considered are the plasma volume (301), the red blood cells (302), the interstitial volume of the organ tissue (304) and the cell volume of the organ tissue (306). Red blood cells and cells of the organ tissue are enclosed by membranes (303), (305), through which the active-agent molecules must permeate. For the compartments enclosed by membranes, permeation terms according to Fick's 2^{nd} law need to be included in the conservation of mass equations for the substance transport. These generally have the form:

$${V}_{x}^{cell} \cdot \frac{ⅆ {C}_{x}^{cell}}{ⅆ t} = P \times {SA}_{x} \cdot ( {C}_{x}^{pl} - \frac{{C}_{x}^{cell}}{{K}_{x}} ) \qquad ( Equation 2 )$$

Vₓ=cell volume of the organ X
Cₓ^{pl} =concentration of the unbound substance in the blood plasma
Cₓ^{cell}=concentration of the substance in the cells of the organ
Kₓ=distribution coefficient of the substance between blood and organ X in the equilibrium state
PxSAₓ=permeability surface-area product for the organ x

The active processes of metabolism and active transport may, for example, be accounted for by so-called Michaelis-Menten terms, which describe the kinetics of the biochemical reactions. An inclusion of the active transport presupposes a permeation-limited model, as described above. A detailed organ model, inclusive of the active processes, is represented in FIG. 4. One or more metabolism processes (401) lead to a reduction in the concentration of the original substance. The active transport processes (402), (403) are described such that they lead to the transport of active-agent molecules across the cell membrane, in parallel with the passive permeation process. For these processes, it should be remembered that distinction needs to be made between being directed inwards (402) and directed outwards (403). Equation 2 is to be modified as follows according to FIG. 4.

$${V}_{x}^{cell} \cdot \frac{ⅆ {C}_{x}^{cell}}{ⅆ t} = P \times {SA}_{x} \cdot ( {C}_{x}^{pl} - \frac{{C}_{x}^{cell}}{{K}_{x}} ) + \frac{{v}_{max}^{in}}{( {k}^{in} + {C}_{x}^{pl} )} \cdot {C}_{x}^{pl} - \frac{{v}_{max}^{out}}{( {k}^{out} + {C}_{x}^{cell} )} \cdot {C}_{x}^{cell} - \frac{{v}_{max}^{metabol}}{( {k}^{metabol} + {C}_{x}^{cell} )} \cdot {C}_{x}^{cell} \\ \text{Here:} \\ \frac{{v}_{max}^{in}}{( {k}^{in} + {C}_{x}^{pl} )} \cdot {C}_{x}^{pl} = Michaelis \text{-} Menten term for describing the kinetics of the inwards transport . \\ \frac{{v}_{max}^{out}}{( {k}^{out} + {C}_{x}^{cell} )} \cdot {C}_{x}^{cell} = Michaelis \text{-} Menten term for describing the kinetics of the inwards transport . \\ \frac{{v}_{max}^{metabol}}{( {k}^{metabol} + {C}_{x}^{cell} )} \cdot {C}_{x}^{cell} = Michaelis \text{-} Menten term for describing the kinetics of the metabolism . \\ {v}_{max}^{y} = maximum rate of process y \\ {k}^{y} = binding constant of the active agent to the protein which is responsible for the process y \\ {V}_{x} = cell volume of the organ X \\ {C}_{x}^{pl} = concentration of the unbound substance in the blood plasma \\ {C}_{x}^{cell} = concentration of the substance in the cells of the organ \\ {K}_{x} = distribution coefficient of the substance between blood and organ X in the equilibrium state \\ {PxSA}_{x} = permeability surface \text{-} area product for the organ x \qquad ( Equation 3 )$$

Organs with more specialized functions are also described according to the same principle, for example the gastrointestinal tract, the kidneys or the biliary tract. Additional parameters, which describe the special physiological functions, generally need to be taken into account for this. In the case of the intestine, the local variation in quantities such as PxSA and pH of the intestinal content also need to be taken into account.

The conservation of mass equations, of the type represented by way of example in Equations 1-3, for the individual compartments and subcompartments are interconnected though the concentrations Cx according to the diagram in FIG. 2. This gives rise to a system of dependent differential equations in time, which can be numerically solved for predetermined initial values. The solutions of this system of equations give the concentration-time relationships for all the compartments contained in the model.

In order to describe a pharmacological effect, the concentration-time relationship in the compartment which contains the biological target of the active agent may furthermore be linked with a pharmacodynamic effect. Typical effect functions are, for example:
Hyperbolic or sigmoid Emax models:

$$Effect = {E}_{0} + \frac{{E}_{max} {C}_{x}^{γ}}{{EC}_{50}^{γ} + {C}_{x}^{γ}}$$
Effect=pharmacological effect parameter (time-dependent)
E₀=base value of the pharmacological effect parameter
Eₘₐₓ=maximum value of the pharmacological effect
EC₅₀=concentration at which 50% of the maximum effect is achieved
Cₓ=concentration at the site of action (time-dependent)
γ=form parameter
Exponential functions: Effect=E₀+βCₓ^{γ} or log-linear model:
   Effect=E₀+βLn(Cₓ)
   Effect=pharmacological effect parameter (time-dependent)
   E₀=base value of the pharmacological effect parameter
   β=parameter for increasing the effect as a function of the concentration
   Cₓ=concentration at the site of action (time-dependent)
   γ=form parameter
Active-agent interaction models, for example partial or complete antagonism, etc.
Combinations of the aforementioned models, with which, for example, multiple action centres or receptor-transducer interactions can be described.

The mode of operation of the overall system for individual dose calculation is then as follows. First, it is necessary to determine the individual values of the parameters of the PBPK/PD model which depend on the physiology or anatomy. To that end, the results of the gene test (101) are evaluated and the proteins for which deviation from the normal population is to be expected in respect of expression or function are identified (102). For these proteins, the expression or the effectiveness is then calculated for the relevant organs by using known and stored relations, and the vₘₐₓ and kₘ values are correspondingly calculated. From the other patient data which are entered, for example body weight, body fat content, sex, age and optionally clinical picture, the parameters V, Q, K, PxSA as well as other parameters necessary for describing special organs, such as the gastrointestinal tract, kidneys, etc., are determined with the aid of the associated relations stored in the knowledge database (105). For this, the standard values of the active agent-specific parameters stored in the active-agent database (107) are taken into account, and these are then modulated according to the individual situation. The genetic or pathologically induced effect on properties such as the composition of the cell membranes and pH values of individual compartments may also need to be taken into account, if these can also affect the permeabilities, PxSA, and the distribution coefficients K.

Once the individual parameter set has been determined, simulation of the pharmacokinetics of the active agent to be administered is carried out with the standard dosing. According to active agent- and therapeutic effect-dependent rules, which are also stored in the knowledge database, a decision is then made as to whether adaptation of the dose is necessary, by using the calculated concentration profiles and optionally the pharmacodynamic effect resulting from them. If it does need to be adapted, a more suitable dose is proposed. This is determined by linear extrapolation for the optimum dose to be achieved in the body, if the dose-linear regime of pharmacokinetics or pharmacodynamics is applicable. If this is not the case, the dose is matched to the optimum by automatically altering it stepwise in the simulation. The result of these optimizations is output from the system and can then be used to adjust the dose.

### BRIEF DESCRIPTION OF THE DRAWINGS AND TABLES

FIG. 1: Basic structure of the overall system for determining the individual dose.

FIG. 2: Flow chart of the structure of the physiology-based pharmacokinetic (PBPK) simulation model.

FIG. 3: Composition of an organ in the PBPK model.

FIG. 4: Principle of the description of active transporters and metabolism processes in the PBPK model.

FIG. 5: Simulated concentration-time curve (line) in blood plasma of patients with CC polymorphism in exon 26 (C3435T) of the MDR1 gene compared with experimentally determined values (points).

FIG. 6: As FIG. 5 plus concentration curve for patients with TT polymorphism in exon 26 (C3435T) of the MDR1 gene (grey line).

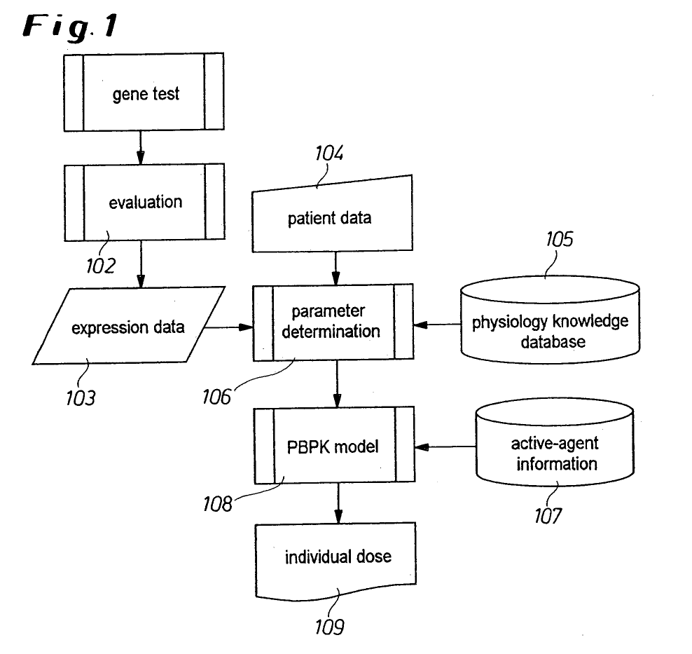

Table 1: Experimental Cmax values and Cmax values determined by simulation for administration of 0.25 mg digoxin in patients with CC and TT polymorphisms in exon 26 (C3435T) of the MDR1 gene.

Table 2: Organ volumes and blood flow rates.

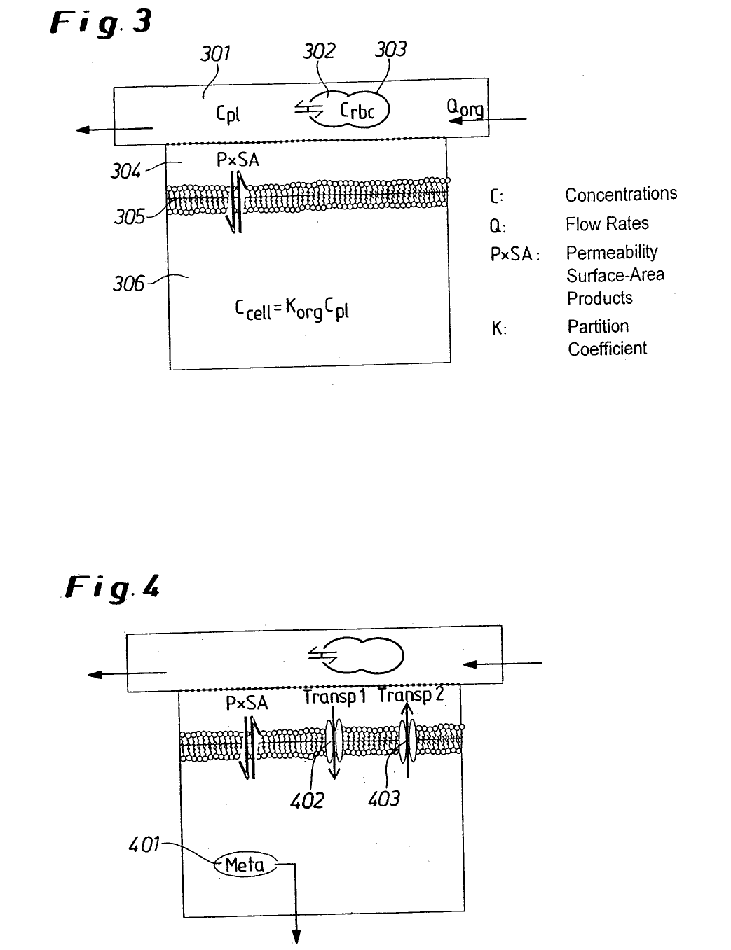

Table 3: Composition of the organs according to FIG. 3.

Table 4: Substance-dependent parameters for digoxin.

Table 5: Organ-specific substance-dependent parameters for digoxin

Table 6: Michaelis-Menten constants for P-gp in the intestinal wall

### EXAMPLE

A non-limiting example which shows how the influence of genetic predisposition for active transporters on the pharmacokinetics of active agents can be described by simulations will be discussed below.

The transport protein which has been most widely studied and described in the literature is p-glycoprotein (P-Gp) which, besides other organs, is expressed particularly in the gut where it can have an influence on the absorption of orally applied active agents. The associated gene is generally identified as MDR1. MDR1 may be present in different alleles, it being known that these lead to different activity of the associated protein (see Martin F. Fromm, The influence of MDR1 polymorphisms on P-glycoprotein expression and function in humans, Advanced Drug Delivery Reviews 54, 1295 (2002)). For P-Gp, tables of pharmaceutical active agents which are a substrate for it have been published (see for example C. J. Matheny et al., Pharmacokinetic and Pharmacodynamic Implications of P-glycoprotein Modulation, Pharmacotherapy, 21, 778 (2001)). One example of such a substrate is digoxin. For digoxin, it is known that its oral absorption depends on which allele of the MDR1 gene is present (see S. Hoffmeyer et al., Proc. Of the National Academy of Science USA, 97, 3473 (2000)).

Concentration-time curves of digoxin in blood plasma for patients with a normally functional MDR1 sequence have been published in A. Johne et al., Clinical Pharmacology & Therapeutics, 66, 339 (1999).

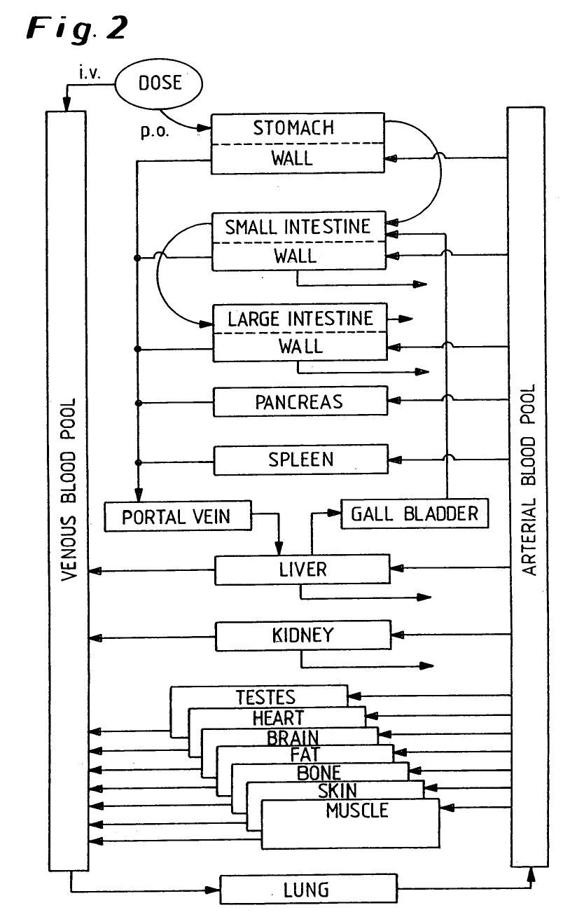

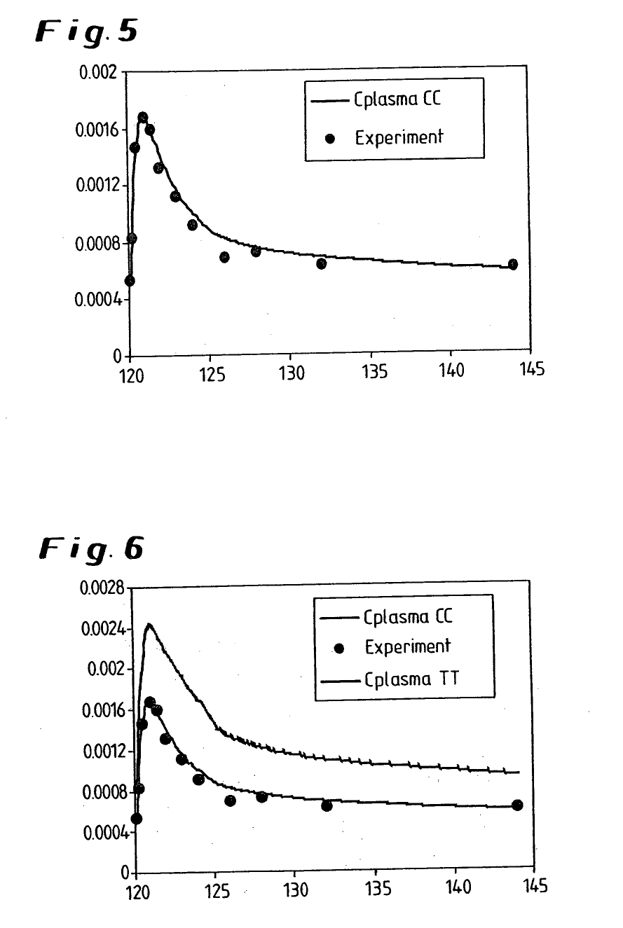

A PBPK model corresponding to FIG. 2, with which these concentration curves are described by simulation, was set up. In this model, besides the passive permeability of the intestinal wall, an active transport process in the direction of the intestinal lumen was also included for the absorption of the substance from the intestine. When using the parameter values listed in Tables 2-6, the simulation model delivers an almost exact description of the experimentally determined plasma-concentration curve (FIG. 5).

In the case in point, the parameter set given in Tables 2-6 is stored in the database of active-agent information (107). The influence of, for example, differing MDR1 sequences can be estimated by correspondingly altering the parameters of the simulation model which are affected by this. This change is made while taking into account the expression data (103) in the “parameter determination” step (106).

An example of a data record in which the expression level of P-Gp in the intestinal wall was determined as a function of the MDR1 polymorphism can be found in S. Hoffmeyer (2000). The expression level in turn determines the maximum rate Vmax of the transport process. In the case in point, the knowledge database (105) hence contains the allocation of relative Vmax values to the gene sequences of the various polymorphisms, which are converted with the substance-specific absolute Vmax values from the database of active-agent information (107) into the parameters to be used in the PBPK model (108).

S. Hoffmeyer et al., (2000) contains additional data about the influence of various polymorphisms on the pharmacokinetics of digoxin. For instance, the Cmax values (maximum plasma concentration) listed in Table 1 are given for the polymorphism in exon 26 (C3435T).

On the basis of the simulation presented above for the “wild type” (C3435T), the homozygotic type TT can be simulated by reducing Vmax by 51% according to the lower expression level. The corresponding result is presented in FIG. 6. The 45% increase of Cmax obtained in the simulation corresponds well to the experimentally found increase of 30% (see Table 1).

In the case in point, Cmax values resulting from the simulation of type TT were compared with safety-critical values contained in the database of active-agent information (107), and a reduced dose was proposed where appropriate. In order to establish the dose proposal, for example, simulations are carried out with iteratively varied doses until the pharmacokinetic characteristics lie in the safe range. In cases in which there is sure to be a linear dependency on the dose, the dose proposal may also be determined by linear extrapolation.

TABLE 1

| | Average relative | Cmax experiment | Cmax simulation |
|---|---|---|---|
| Polymorphism | expression level | [μg/l] | [μg/l] |
| CC (wild type) | 1275 | 1.6 | 1.72 |
| TT | 627 | 2.1 | 2.49 |
| (homozygotic) | | | |

TABLE 2

| | Volume | Blood flow rate |
|---|---|---|
| Organ | [ml] | [ml/min] |
| Venous blood pool | 250 | 4670 |
| Arterial blood pool | 140 | 4670 |
| Lung | 670 | 4670 |
| Stomach | 150 | 60 |
| Small intestine | 640 | 600 |
| Large intestine | 370 | 240 |
| Pancreas | 100 | 60 |
| Spleen | 180 | 180 |
| Liver | 1710 | 390 |
| Gall bladder | 20 | |
| Kidney | 720 | 1133 |
| Brain | 1486 | 700 |
| Heart | 330 | 240 |
| Muscle | 30200 | 550 |
| Bone | 12060 | 167 |
| Skin | 3020 | 50 |
| Fat | 10060 | 300 |
| Testes | 35 | 2.6 |

TABLE 3

| Volume fraction | f_vas | f_int | f_cell |
|---|---|---|---|
| Fat tissue | 0.010 | 0.135 | 0.855 |
| Brain | 0.037 | 0.004 | 0.959 |
| Gastrointestinal tract | 0.032 | 0.100 | 0.868 |
| Heart | 0.262 | 0.100 | 0.638 |
| Kidney | 0.105 | 0.200 | 0.695 |
| Liver | 0.115 | 0.163 | 0.722 |
| Lung | 0.626 | 0.188 | 0.186 |
| Muscle | 0.026 | 0.120 | 0.854 |
| Bone | 0.041 | 0.100 | 0.859 |
| Skin | 0.019 | 0.302 | 0.679 |
| Pancreas | 0.180 | 0.120 | 0.700 |
| Spleen | 0.282 | 0.150 | 0.568 |
| Testes | 0.140 | 0.069 | 0.791 |

TABLE 4

| Parameter | Value | Unit |
|---|---|---|
| Dose | 0.25 | mg |
| Water solubility | 65 | mg/l |
| Intrinsic liver clearance | 0.48 | ml/min/kg |
| Free fraction in plasma | 0.73 | — |
| Passive intestine wall permeability | 1.4 10⁻⁶ | cm/s |

TABLE 5

| | Organ/plasma | Permeability surface- |
|---|---|---|
| Organ | distribution coefficient | area product [ml/min] |
| Stomach | 8.34 | 100 |
| Small intestine | 8.34 | 1000 |
| Large intestine | 8.34 | 500 |
| Pancreas | 10.57 | 500 |
| Spleen | 2.74 | 250 |
| Liver | 9.35 | 1000 |
| Kidney | 7.19 | 1000 |
| Lung | 1.97 | 0.3 |
| Brain | 14.21 | 0.002 |
| Heart | 13.28 | 1000 |
| Muscle | 2.33 | 1000 |
| Bone | 34.45 | 28 |
| Skin | 13.49 | 0.33 |
| Fat | 100.42 | 14 |
| Testes | 4.42 | 3 |

TABLE 6

| | Vₘₐₓ | Km |
|---|---|---|
| Organ | [ml/min] | [mg/l] |
| Duodenum | 0.086 | 6.3 |
| Jejunum | 0.76 | 6.3 |
| Ileum | 1.13 | 6.3 |

The foregoing is only a description of a non-limiting number of embodiments of the present invention. It is intended that the scope of the present invention extend to the full scope of the appended issued claims and their equivalents.

1. A method for determining a dose of at least one active agent based partially on a genetic analysis of a patient or subject, the method comprising the following steps:
a) analyzing the sequence and/or expression of at least one gene in a patient,
b) assigning the at least one gene to physiological functions of a human or animal body, wherein the physiological functions influence at least one pharmacokinetic parameter selected from the group consisting of metabolism, absorption, excretion and distribution of the active agent in the body,
c) inputting patient data,
d) calculating relevant physiological parameters for the PBPK model from the patient data from b) and c) by integrating additional information contained in a knowledge database, and delivering the parameters to the PBPK model,
e) inputting active agent-specific data into the PBPK model, directly or from a database
f) determining the optimal individual dose of the at least one active agent by simulating the pharmacokinetic profile of the at least one active agent and adjusting the dose for best fit to optimal profile.

2. The method of claim 1, wherein the at least one gene is related to at least one protein selected from the group consisting of metabolizing enzymes selected from the group consisting of monooxygenases of the cytochrome P 450 family, phase II enzymes which attach polar groups to the molecules to be excreted, active transporters, multidrug resistance proteins, plasma binding proteins, serum albumin and glycoproteins.

3. The method of claim 2, wherein at least one multidrug resistance protein is selected from the group consisting of the P-glycoprotein family, multidrug resistance-associated proteins (MRP), the organic anion transporting polypeptide family (OATP) the organic anion transporter family (OAT), the organic cation transporter family (OCT), the novel organic cation transporter family (OCTN), and the peptide transporter family (PepT).

4. The method of claim 1, wherein the active agent-specific data are those selected from the group consisting of organ/blood distribution coefficients, membrane permeability, kinetic constants of metabolism processes and active transport processes.

5. The method of claim 1, wherein the patient data are selected from the group consisting of body weight, body surface area, body fat content, age and sex.

6. The method of claim 1, wherein the physiological parameters according to step f) are selected from the group consisting of flow rate Qₓ of blood through an organ X, volume Vₓ of the organ X and permeability surface-area product (PxSAₓ) for the organ X.

7. The method of claim 1, wherein the PBPK model is a simulation program which simulates at least one function selected from the group consisting of intestinal absorption, blood transport, distribution in organs by permeation or active transport, metabolism, and excretion through urine or bile.

8. The method of claim 1, wherein the analyzing of the at least one gene comprises the use of a gene sequence specific sensor.

9. The method of claim 1, wherein the analyzing of the at least one gene comprises quantitatively determining the presence of gene-specific RNA molecules.

10. The method of claim 1, wherein the analyzing of the at least one gene comprises determining the presence of gene-specific protein molecules.

11. A device for determining the dose of active agents by performing the method of claim 1, the device comprising:
a) at least one gene sequence-specific analysis instrument; and
b) a computer unit connected to the analysis instrument and comprising a program comprising a pharmacokinetic model, a knowledge database and input modules for patient data, wherein the pharmacokinetic model is a physiology-based pharmacokinetic model (PBPK model).

12. A method for determining a dose of at least one active agent by simulating a patient's pharmacokinetic profile, the method comprising the following steps:
a) analyzing the sequence and/or expression of at least one gene in a patient, wherein the gene's expression is characterized in terms of gene-specific RNA or gene-specific protein levels,
b) allocating the at least one gene to physiological functions of a human or animal body, wherein the physiological functions influence at least one pharmacokinetic parameter selected from the group consisting of metabolism, absorption, excretion and distribution of the active agent in the body,
c) inputting patient-specific data relevant to the physiological state of the patient,
d) determining the parameters relevant to calculating a dose of an active agent by combining the results of steps a), b) and c) with knowledge database entries describing the physiologic and kinetic behavior of the at least one gene-specific protein,
e) applying the results of step d) to a physiology-based pharmacokinetic model that further comprises pharmacokinetic data for at least one active agent, and
f) determining the optimal individual dose of the at least one active agent by simulating the pharmacokinetic profile of the at least one active agent and adjusting the dose for best fit to optimal profile.
